(19) United States
(12) Patent Application Publication
(10) Pub. No.: US 2024/0377346 A1
(43) Pub. Date: Nov. 14, 2024

(54) HAEMATOCRIT AND HAEMOGLOBIN DETECTION USING CAPACITANCE

(71) Applicant: OSLER DIAGNOSTICS LIMITED, Oxford (GB)

(72) Inventors: Brian Peter HARROP, Oxford (GB); Sean Edward GOODWIN, Oxford (GB); Flavio Cesar Bedatty FERNANDES, Oxford (GB); Alexander Drew TODD, Oxford (GB); Daniel DE BURCA, Oxford (GB); Stanislav SOKOLOV, Oxford (GB); Michael Alexander O'CONNELL, Oxford (GB)

(21) Appl. No.: 18/681,757

(22) PCT Filed: Aug. 4, 2022
(86) PCT No.: PCT/EP2022/071919

§ 371 Date: Feb. 6, 2024

(30) Foreign Application Priority Data
Aug. 6, 2021 (GB) 2111393.1

(51) Int. Cl. G01N 27/22; G01N 33/49

(52) U.S. Cl. CPC G01N 27/228; G01N 33/49

(57) ABSTRACT

A method of determining haematocrit or haemoglobin concentration of a blood sample comprises calculating, based on a plurality of complex capacitance values of the blood sample, a complex capacitance at a predetermined imaginary component value, each of the plurality of complex capacitance values of the blood sample having a corresponding frequency; and determining the haematocrit or haemoglobin concentration based on the calculated complex capacitance or a real component of the calculated complex capacitance.

The present disclosure relates to methods of determining the haematocrit or haemoglobin concentration of a blood sample, a computer-readable medium comprising instructions for determining the same, a system for determining the same and a device configured to determine the same.

### BACKGROUND

Haematocrit is the ratio of erythrocytes to blood plasma in a blood sample. The current industry standard method for the electrochemical measurement of haematocrit comprises measuring the conductivity of a blood sample. The conductivity of blood decreases with increasing haematocrit due to the very low conductivity of the erythrocytes and the relatively high conductivity of the plasma the erythrocytes are suspended in. This is the fundamental principal that the Maxwell-Fricke equation is based upon, which describes the relationship between haematocrit and resistance of blood:

$${R}_{s} = {R}_{p} \frac{ϕ + 2}{2 ( 1 - ϕ )}$$

where Rₛ=resistance of blood/solution, Rₚ=resistance of plasma and ϕ=haematocrit.

Haematocrit is a value which varies between 0 and 1 (optionally expressed as a percentage) for pure plasma and pure erythrocytes, respectively. To obtain the solution resistance, the simplest method is to measure the impedance of solution at a single high frequency to determine the solution resistance whilst reducing the effect of the double layer capacitive effect on the resistance measurement. The Maxwell-Frick method is reliant on an accurate value of plasma resistance to give an accurate haematocrit value, however, the value for plasma resistance depends on electrolyte content and concentration, which includes salts, colloidal electrolytes, proteins and anticoagulants. When these factors vary from normal values, through either clinical condition or clinical procedure, plasma resistance also varies. This can add significant error to the haematocrit value derived via the Maxwell-Fricke method. This reliance on an accurate value of plasma resistance for the Maxwell-Fricke method is an issue for point of care devices. These devices cannot get a plasma resistance measurement for each sample tested and instead use an internally stored average plasma resistance value that, if incorrect, will increase error in the point of care measurement. Therefore, improved methods of measuring haematocrit are needed. Haematocrit is closely correlated with blood haemoglobin concentration, and therefore improved methods of measuring haematocrit can also be used to measure haemoglobin concentration.

Essentially, capacitance is charge stored at an interface due to an applied potential. Without wishing to be constrained by theory, when one electrode is in contact with a solution, interfacial capacitance is given by:

Cᵢ=εᵣε₀k

wherein εᵣ is the relative static permittivity (generally referred to as the dielectric constant) of the liquid, ε₀ is the dielectric constant or dielectric permittivity of vacuum and k is the inverse Debye length (LD=1/k), wherein k is given by k=[(2e²N)/εᵣε₀k_{B}T)]^{1/2}. N is the molar concentration of any polarisable species in the solution, which means that there is a k for each species and the total capacitance is generally the sum of the capacitance of all species. Capacitance is based on the charging/discharging process that can be caused by rearrangement of ions or oscillation of dipoles (in one species) at the electrode/solution interface. In other words, it is the accumulation of charge on the surface of an electrode. Electrode/electrolyte interfaces are complex with different processes resonating at different frequencies. Thus, the complex capacitance depends on the potential applied and the time of perturbation i.e. the frequency of the input signal in an electrochemical impedance spectroscopy (EIS) measurement, for example.

### SUMMARY

This summary introduces concepts that are described in more detail in the detailed description. It should not be used to identify essential features of the claimed subject matter, nor to limit the scope of the claimed subject matter.

In one aspect, a method of determining haematocrit of a blood sample may comprise calculating, based on a plurality of complex capacitance values of the blood sample, a complex capacitance at a predetermined imaginary component value. As would be understood, each of the plurality of complex capacitance values of the blood sample has a corresponding frequency. The corresponding frequency may be the frequency of the input signal used to measure the complex capacitance value. The method may further comprise determining the haematocrit based on the calculated complex capacitance or a real component of the calculated complex capacitance. The method may also be used to determine haemoglobin concentration, mutatis mutandis.

This method may be referred to herein as the ‘capacitance’ method, in contrast to conventional ‘impedance’ methods such as the Maxwell-Fricke method, that take into consideration the resistive component of the impedance of a blood sample in order to determine haematocrit. Unlike the Maxwell-Fricke method, the capacitance method does not necessarily assume a known plasma resistance. As demonstrated below, the capacitance method is found to show a decrease in sensitivity to salts and other donor-to-donor variability compared to the Maxwell-Fricke method, leading to improved haematocrit detection performance.

Calculating a complex capacitance at a predetermined imaginary component value may comprise extrapolating from the plurality of complex capacitance values. Extrapolating may comprise fitting a circular arc to the plurality of complex capacitance values in capacitance space and extrapolating the arc to calculate the complex capacitance at the predetermined imaginary component value. Capacitance space is a plot of the real component of the complex capacitance versus the imaginary component of the complex capacitance. The circular arc fitting may be transformed to a weighted ordinary least squares fitting—an example transformation operation is described below in the detailed description. When fitting an arc, the plurality of complex capacitance values may be at least four complex capacitance values.

The corresponding frequencies, f, have a maximum value, fₘₐₓ, and fitting the circular arc may comprise fitting the arc for the range of frequencies f & fₘₐₓ. Additionally or alternatively, the corresponding frequencies, f, have a minimum value, fₘᵢₙ, and the circular arc is fit for the range of frequencies fₘᵢₙ≤f. The circular arc may be fit by applying an algorithm for least-squares estimation of nonlinear parameters, such as a Levenberg-Marquardt algorithm or damped least squares method. Each corresponding frequency of the plurality of complex capacitance values of the blood sample may be less than or equal to 1000 kHz, less than or equal to 500 kHz, less than or equal to 200 kHz or less than or equal to 100 KHz. Each corresponding frequency of the plurality of complex capacitance values of the blood sample may be greater than or equal to 0.1 Hz, greater than or equal to 10 Hz, or greater than or equal to 1 kHz.

Determining the haematocrit based on the calculated complex capacitance or the real component of the calculated complex capacitance may comprise using a calibration curve. Optionally, the calibration curve is an inverted linear calibration curve:

$$\\ ϕ = \frac{C - α}{β}$$

wherein ϕ is the haematocrit, C is the calculated complex capacitance or the real component of the calculated complex capacitance, α is the intercept and β is the slope.

The calculated complex capacitance or the real component of the calculated complex capacitance may correspond to the capacitance of the blood sample at frequencies tending towards infinity. As the frequency approaches infinity, the imaginary component of the complex capacitance becomes insignificant and so the calculated complex capacitance and the real component of the calculated complex capacitance become substantially equal meaning value either value can be used to reliably determine haematocrit. At frequencies tending towards infinity (e.g. frequencies greater than 100 or 1000 kHz, for example) there is no significant influence from the concentration of ions, reducing the effect of variable salt concentration on the haematocrit measurement. The predetermined imaginary component value may be less than or equal to 500 pF/mm², preferably less than or equal to 200 pF/mm², more preferably less than or equal to 100 pF/mm², and yet more preferably substantially equal to or equal to zero.

In another aspect, a method of determining haematocrit or haemoglobin concentration of a blood sample comprises determining a plurality of complex impedance values of the blood sample, each of the plurality of complex impedance values having a corresponding frequency; calculating a plurality of complex capacitance values of the blood sample based on the plurality of complex impedance values; and performing a method as described above, using the plurality of complex capacitance values. The method may be performed on whole blood or lysed blood.

In another aspect, a computer-readable medium comprises instructions which, when executed by one or more processors, causes the one or more processors to perform any of the methods described above.

In another aspect, a system for determining haematocrit or haemoglobin concentration of a blood sample comprises a cell configured to receive the blood sample; a device configured to determine a plurality of complex impedance values of the blood sample; a computer-readable medium as described above; and a processor configured to execute the instructions of the computer-readable medium. As would be understood, execution of the instructions of the computer-readable medium is based on the plurality of complex impedance values, from which the corresponding complex capacitance values are determined. The processor may be further configured to calculate the plurality of complex capacitance values of the blood sample based on the plurality of complex impedance values.

In another aspect, a device configured to determine haematocrit or haemoglobin concentration of a blood sample comprises two electrodes. A distance between the electrodes may be less than or equal to 2 mm, 1 mm or 0.5 mm. The two electrodes may be arranged such that when the electrodes are submerged in blood plasma, a solution resistance of the blood plasma may be less than 20 kΩ, less than 10 kΩ, or less than 5 kΩ. A track resistance of a track between one of the electrodes and a corresponding electrode contact may be less than or equal to 1 kΩ, 0.7 kΩ or 0.5 kΩ. The electrodes may be in a cell configured to receive the blood sample. The device may further comprise electronics, such as a potentiostat, configured to determine the impedance of the blood sample at a plurality of frequencies. The device may comprise additional electrodes, such as a reference electrode. The cell may have an internal volume of 1 to 20 μL. The device may be for making measurements in vitro i.e. outside of the human or animal body. The device may not be suitable for in vivo implantation. The device may be integrated into the above-described system and/or the cell of the above-described system may have electrodes with the above-described configuration.

### BRIEF DESCRIPTION OF THE DRAWINGS

Embodiments of the present invention will now be described, by way of example only, with reference to the accompanying drawings, in which:

FIG. 1 illustrates a Nyquist plot of complex capacitance data for a blood sample with unknown haematocrit;

FIGS. 2a and 2b illustrate a method for determining haematocrit of a blood sample;

FIG. 3 illustrates a system for determining haematocrit of a blood sample;

FIGS. 4a-c illustrate capacitance data for blood plasma and whole blood with 65% haematocrit, where FIG. 4a is a Nyquist plot and FIGS. 4b and 4c are corresponding Bode plots for the real and imaginary capacitance, respectively;

FIGS. 5a-d illustrate haematocrit detection performance for fixed sodium concentration, where FIG. 5a is a Nyquist plot of derived capacitance fitted circular arcs for different haematocrit concentrations;

FIG. 5b is a calibration plot demonstrating observed haematocrit ϕ versus estimated intercept of the axis of the real component of the complex capacitance C′_{intercept}, including the linear regression slope (black line) and 95% prediction interval (grey shading); FIG. 5c is a plot of observed haematocrit ϕ versus haematocrit recovered using the Maxwell-Fricke method, including the linear regression slope (solid line) and the 1:1 line (dashed line); and FIG. 5d is as in FIG. 5c, except that haematocrit ϕ is recovered via the C′_{intercept} calibration (capacitance method);

FIGS. 5e and 5f relate to haemoglobin concentration determination, where FIG. 5e is a calibration plot demonstrating observed haemoglobin concentration versus estimated intercept of the axis of the real component of the complex capacitance C′_{intercept}, including the linear regression slope (black line) and 95% prediction interval (grey shading) and FIG. 5f is a plot of observed haemoglobin concentration versus haemoglobin concentration recovered via the C′_{intercept} calibration (capacitance method), including the linear regression slope (solid line);

FIG. 6 illustrates haematocrit detection error analysis for fixed sodium concentration, where FIG. 6a is a series of histograms demonstrating the distribution of haematocrit ϕ recovery error for the Maxwell-Fricke and C′_{intercept} calibration (capacitance) methods; FIG. 6b is haematocrit ϕ recovery error from the Maxwell-Fricke method versus observed haematocrit ϕ, with the mean error +/−1.96 standard deviations denoted (dashed lines) alongside the p-value from a D'Agostino and Pearson normality test; and FIG. 6c is as in FIG. 6b, except that haematocrit ϕ recovery errors are from the C′_{intercept} calibration (capacitance method);

FIGS. 7a-d are as in FIGS. 5a-d, expect for varying Na⁺ concentration spikes;

FIGS. 8a-c are as in FIG. 6a-c, expect for varying Na⁺ concentration spikes;

FIG. 9 illustrates a cell for performing an impedance measurement on a blood sample;

FIG. 10 illustrates a device configured to determine haematocrit of a blood sample;

FIG. 11a illustrates a calibration curve for the capacitance method using an electrode spacing of 2.4 mm; and

FIG. 11b illustrates a calibration curve for the capacitance method using an optimised cell design with electrode spacing of 0.6 mm.

### DETAILED DESCRIPTION

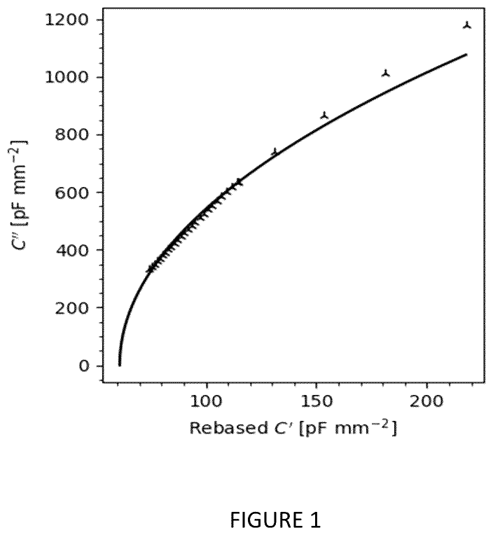

FIG. 1 is a Nyquist plot of complex capacitance data (i.e. a plot in capacitance space) for a blood sample with unknown haematocrit. The vertical axis corresponds to the imaginary component of the complex capacitance, C″, and the horizontal axis corresponds to the real component of the complex capacitance, C′.

Each complex capacitance value has been derived from the complex impedance of the blood sample measured at a specific frequency and thus also has a corresponding frequency. In FIG. 1, the frequencies range from 200 kHz (left-most point for each blood sample) to 25 Hz (right-most point for each blood sample), with 25 points per decade. The lowest frequency is termed the minimum frequency level, fₘᵢₙ.

The impedance measurements were performed using a conventional potentiostat (EMStat Pico, Palmsens BV, Houten, The Netherlands). The amplitude of the input signal was 10 mV about a potential of 0.0 V vs screen-printed carbon electrode (SPCE). The blood sample volume was 10 μl and measurements were taken at 30° C. The dimensions of the flow cell were 2.4 mm by 9.7 mm by 225 μm. The electrodes were rectangular electrodes with dimensions of 0.3 mm by 2 mm with a separation of 0.6 mm. In the Figures, the capacitances are normalised for electrode area, although this is not essential. Each impedance measurement is taken at a specific frequency (i.e. at one of the above described plurality of frequencies). In the Figures, the C′ axis is rebased by adding a constant (−1*minimum extrapolated C′ for 0% haematocrit, for example as determined during calibration-discussed below) in order to normalise the data. This does not affect the final haematocrit results and is not an essential part of the capacitance method.

The complex impedance measurement result consists of two values: the real part, Z′, and the imaginary part, Z″. The complex impedance measurement result is used to calculate C′ and C″ of the complex capacitance using the following formulae:

$${C}^{′} = \frac{- {Z}^{″}}{2 π f {\vert Z \vert}^{2}}$$

$${C}^{″} = \frac{{Z}^{′}}{2 π f {\vert Z \vert}^{2}}$$

Where f is the respective frequency and |Z| is the magnitude of the vector of the real and imaginary components of the complex impedance in the complex plane.

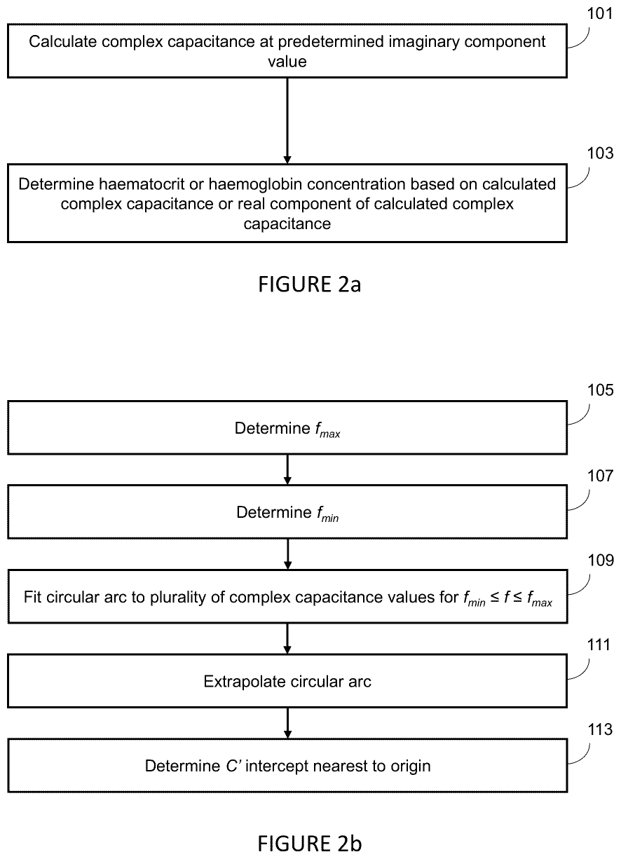

FIG. 2a illustrates a method of determining the haematocrit of a blood sample. The method comprises calculating 101, based on a plurality of complex capacitance values of the blood sample, a complex capacitance at a predetermined imaginary component value, each of the plurality of complex capacitance values of the blood sample having a corresponding frequency, as explained above.

In the present example, the plurality of complex capacitance values of the blood sample are the complex capacitance values of the points in FIG. 1. The predetermined imaginary component value is C″=0 pF mm⁻², also known as the intercept of the horizontal axis or C′ intercept.

The method of FIG. 2a further comprises determining 103 the haematocrit based on the calculated complex capacitance or a real component of the calculated complex capacitance, which is discussed later. Alternatively or additionally, haemoglobin concentration may be determined, which is also discussed later.

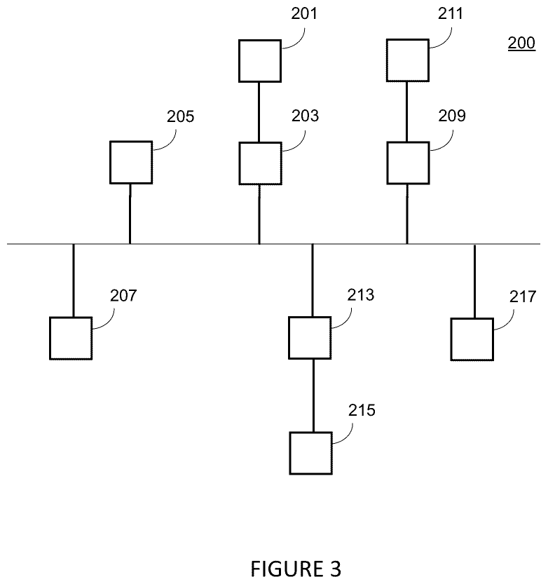

With reference to FIG. 3b, in the present example, calculating 101 a complex capacitance at a predetermined imaginary component value comprises determining 105 the maximum frequency value, fₘₐₓ, of the frequencies corresponding to the plurality of complex capacitance values. The minimum frequency value, fₘᵢₙ, of the frequencies corresponding to the plurality of complex capacitance values is also determined 107.

A circular arc is fitted 109 to the plurality of complex capacitance values in capacitance space for the range of frequencies fₘᵢₙ≤f≤fₘₐₓ. That is, for the range of frequencies fₘᵢₙ≤f≤fₘₐₓ, the circular arc which satisfies:

$${( {C}^{′} - A )}^{2} + {( {C}^{′′} - B )}^{2} = {R}^{2}$$

is fitted 109, such that R>0, A≥0 and B≤0.

Initial guesses for A and B are the median of C′ and C″, respectively. As an initial guess for R, half the Euclidean distance between the (C′; C″) points at the minimum and maximum values of C′ is used.

Let

$$δ = δ ( f ) = [ ( {{C}^{′} ( f )}^{2} + {{C}^{″} ( f )}^{2} ) ] - [ {R}^{2} - ( {A}^{2} + {B}^{2} ) + 2 ( {AC}^{′} ( f ) + {BC}^{″} ( f ) ) ]$$

represent a nonlinear cost function.

Let

$$w = w ( f ) = {( \frac{f}{{f}_{max}} )}^{2}$$

represent a quadratic weighting function.

The circular arc is fitted 109 by applying the iterative Levenberg-Marquardt nonlinear least squares algorithm (an algorithm for least-squares estimation of nonlinear parameters) to find optimal estimates for A, B and R which minimise the weighted sum of squares:

$${\sum }_{{f}_{i} = {f}_{min}}^{{f}_{max}} {( δ ( {f}_{i} ) w ( {f}_{i} ) )}^{2} .$$

The fitted circular arc is extrapolated 111 to determine 113 the C′ intercept nearest to the origin, which represents the theoretical capacitance as frequency tends towards too:

$${C}_{i n t e r c e p t}^{′} = {min}_{\{ x : {C}^{″} ( x ) = 0 \}} {C}^{′} ( x ) = A - \sqrt{{R}^{2} - {B}^{2}}$$

That is, the predetermined imaginary component value in this example is 0 pF mm⁻². The fitted and extrapolated arc is shown in FIG. 1.

As would be understood, other predetermined imaginary component values close to 0 pF mm⁻² could also be used, and both the complex capacitance or a real component of the complex capacitance at this point could be used, since these will have similar values to the C′ intercept nearest to the origin.

Furthermore, an equivalent, unphysical estimator would be given by the C′ intercept furthest from the origin:

$${C}_{{intercept}^{*}}^{′} = {max}_{\{ x : {C}^{″} ( x ) = 0 \}} {C}^{′} ( x ) = A + \sqrt{{R}^{2} - {B}^{2}}$$

In the present disclosure, only results from the physical estimator are presented, but identical recovery performance would be achieved using the unphysical estimator.

The parameter B may be assumed to be equal to zero in order to simplify the fitting process, in which case B may be excluded from the equations above.

Optionally, the circular arc fitting may be transformed to a weighted ordinary least squares fitting:
For N complex capacitance values each with a real component C′ and imaginary component C″ and corresponding frequency f, wherein fₘₐₓ is the maximum frequency of the frequencies corresponding to the N complex capacitance values, transformed variables X and Y, and fitting weights, W, are calculated:

$$X = \{ {C}_{i}^{′} \text{:} i = 1 , 2 , … , N \}$$

$$Y = \{ {{C}_{i}^{′}}^{2} + {{C}_{i}^{′′}}^{2} \text{:} i = 1 , 2 , … , N \}$$

$$W = \{ {( \frac{{f}_{i}}{{f}_{max}} )}^{2} \text{:} i = 1 , 2 , … , N \}$$

Weighted ordinary least squares is then applied to estimate the maximum likelihood fit through (X, Y) subject to weights W:

$$\overline{{X}_{W}} = \frac{{\sum }_{i = 1}^{N} {W}_{i} {X}_{i}}{{\sum }_{i = 1}^{N} {W}_{i}} \\ \overline{{Y}_{W}} = \frac{{\sum }_{i = 1}^{N} {W}_{i} {Y}_{i}}{{\sum }_{i = 1}^{N} {W}_{i}} \\ \hat{β} = \frac{{\sum }_{i = 1}^{N} {W}_{i} ( {X}_{i} - \overline{{X}_{W}} ) ( {Y}_{i} - \overline{{Y}_{W}} )}{{\sum }_{i = 1}^{N} {{W}_{i} ( {X}_{i} - \overline{{X}_{W}} )}^{2}}$$

$$\hat{α} = \overline{{Y}_{W}} - \hat{β} \overline{{X}_{W}}$$

Parameters A and R are then estimated using:

$$A = \frac{\hat{β}}{2}$$

$$R = \sqrt{\hat{α} + {( \frac{\hat{β}}{2} )}^{2}}$$

In this example, parameter B is set to zero.

Advantageously, using this or a similar transformation may allow the C′ intercept to be calculated using fewer computational resources or less powerful computational software.

Referring back to FIG. 2a, in the present example, the step of determining 103 the haematocrit based on the calculated complex capacitance or the real component of the calculated complex capacitance comprises using a haematocrit calibration curve, wherein the calibration curve is an inverted linear calibration curve:

$$ϕ = \frac{C - α}{β}$$

wherein ϕ is the haematocrit, C is the C′ intercept nearest to the origin, α is the intercept and β is the slope. Haemoglobin concentration can be determined using a haemoglobin concentration calibration curve instead.

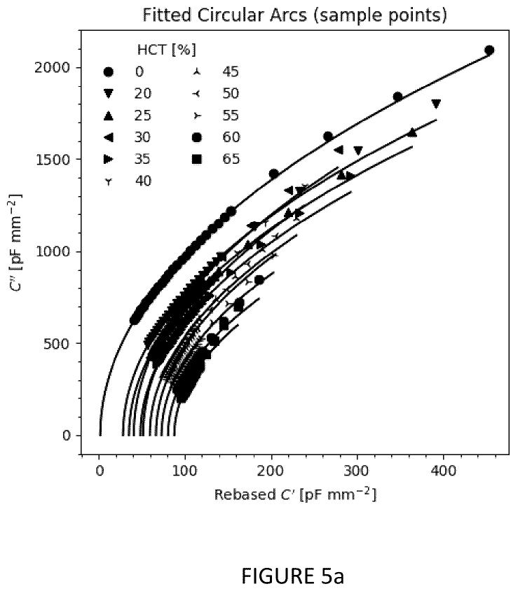

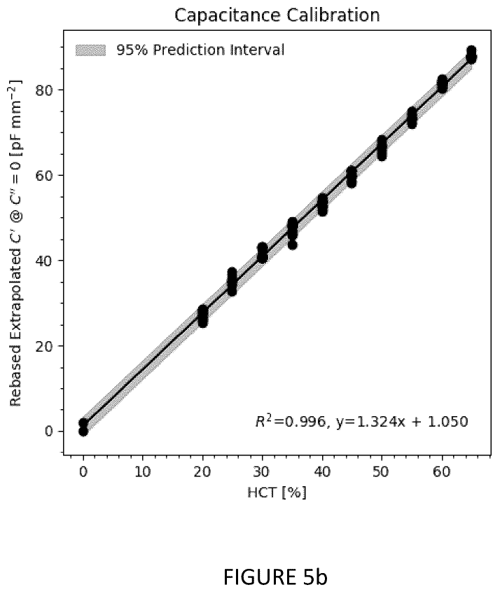

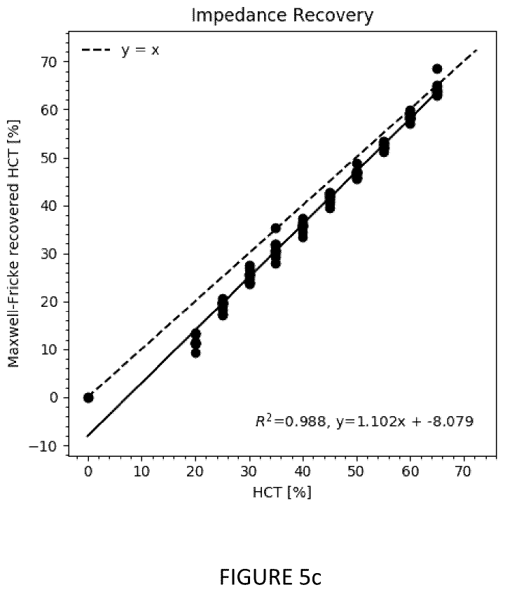

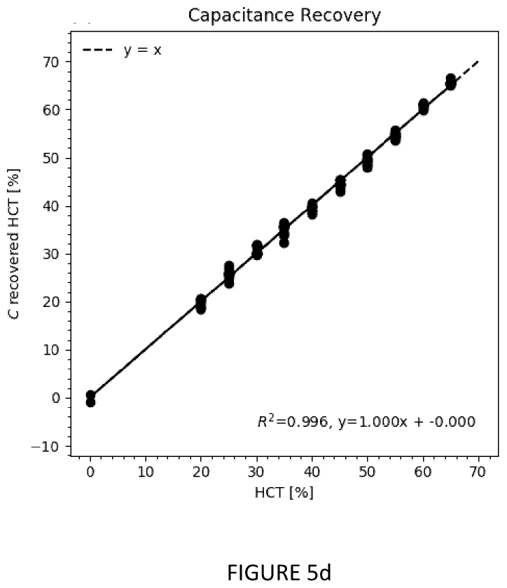

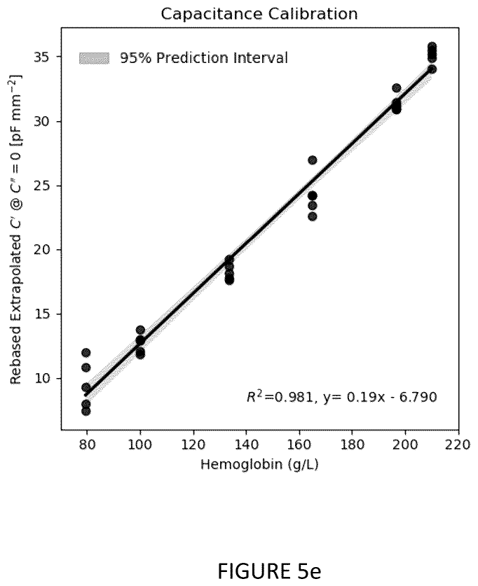

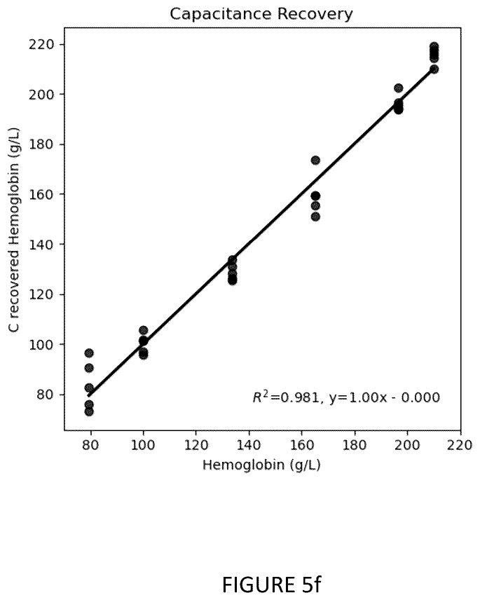

The constants α and β are calculated in a conventional manner by determining the C′ intercept values for a plurality of blood samples with known haematocrit values and calculating the corresponding α and β values using a (linear) least squares best fit. Constants α and β for a haemoglobin concentration calibration curve can be determined in an analogous manner. FIGS. 5a and 5b, discussed below, illustrate sample data for preparing a calibration curve from capacitance data for blood samples with known haematocrit, and the resulting calibration curve. A haemoglobin calibration curve is illustrated in FIG. 5e.

Standard methods such as the microhaematocrit capillary method for determining haematocrit of a blood sample are known to the skilled person. For example, using this method, the blood sample is drawn into a capillary and centrifuged, and then the ratio of erythrocytes to blood plasma (i.e. haematocrit) can be measured and expressed as a decimal or percentage fraction. Blood samples with known haematocrit may be prepared by 1) measuring the haematocrit of a native sample; 2) centrifuging the blood sample; and 3) removing or adding plasma to adjust the haematocrit to the desired value.

A haemoglobin concentration calibration curve can be obtained by taking a blood sample with typical haemoglobin concentration (e.g. 157 g/L) and removing or adding donor plasma to create a range of blood samples with different haemoglobin concentrations. The concentration of each adjusted blood sample is then measured, for example using a HemoCue Hb 201+ system. The samples are then measured using the capacitance method and the C′ intercept plotted against the haemoglobin concentration, for example as illustrated in FIG. 5e.

The described methods may be implemented using computer executable instructions. A computer program product or computer readable medium may comprise or store the computer executable instructions. The computer program product or computer readable medium may comprise a hard disk drive, a flash memory, a read-only memory (ROM), a CD, a DVD, a cache, a random-access memory (RAM) and/or any other storage media in which information is stored for any duration (e.g. for extended time periods, permanently, brief instances, for temporarily buffering, and/or for caching of the information). A computer program may comprise the computer executable instructions. The computer readable medium may be a tangible or non-transitory computer readable medium. The term “computer readable” encompasses “machine readable”. Thus, in an aspect, there is provided a computer-readable medium comprising instructions which, when executed by one or more processors, causes the one or more processors to perform the method of FIGS. 2a and/or 2b.

FIG. 3 is a diagram of a system 200 for determining haematocrit of a blood sample. The system 200 comprises a central bus structure; a cell 201 configured to receive a blood sample; a potentiostat 203 connected to the cell 201 and central bus structure and configured to determine a plurality of complex impedance values of the blood sample in the cell 201; data processing resources such as memory 205 connected to the central bus structure and storing the above-described computer-readable medium; a processor 207 connected to the central bus structure and configured to execute the instructions of the computer-readable medium; and a display adapter 209, display device 211, one or more user-input device adapters 213, one or more user-input devices 215, such as a keyboard and/or a mouse, and one or more communications adapters 217, all connected to the central bus structure. The display device 211 has touch-input functionality and so also functions as a user-input device 215.

The potentiostat may comprise or be in communication with a further processor and further data processing resources for processing measurement data, or may use the memory 205 and processor 207 of the system in order to measure and/or calculate impedance and capacitance data.

### Performance Data

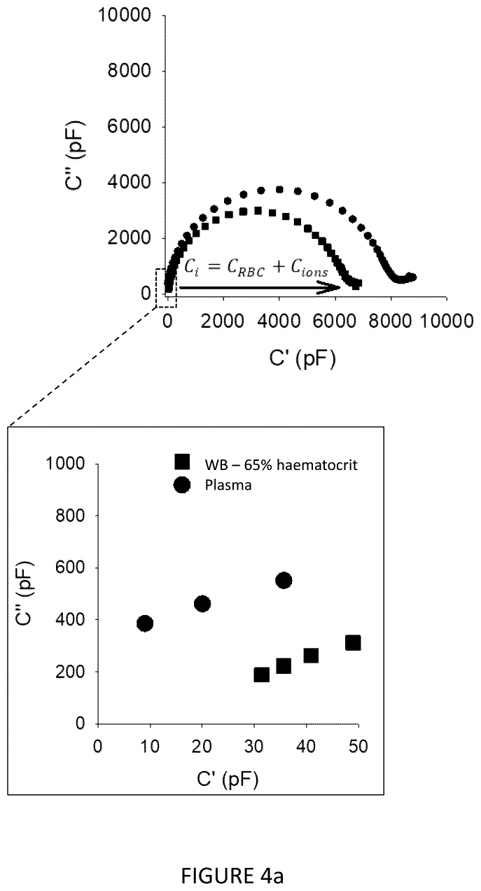

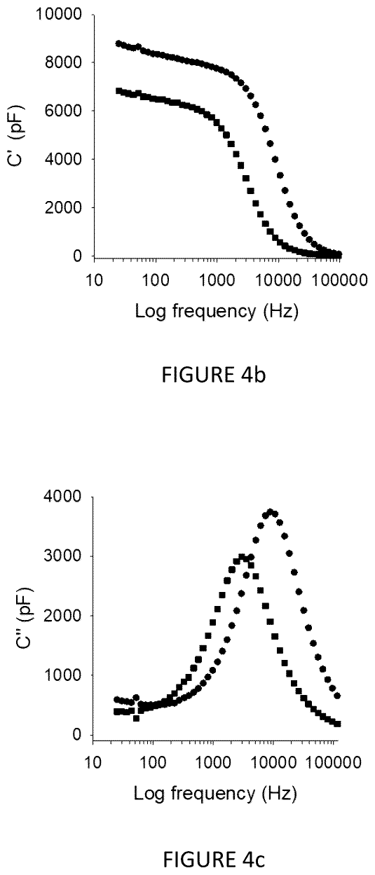

FIG. 4a is a Nyquist plot for impedance-derived capacitance of blood (65% haematocrit, square points) and pure serum/plasma (0% haematocrit, circular points). FIG. 4a includes a close up of the region near the origin. High frequency data points are on the left-hand-side while low frequency measurements are on the right-hand-side. Thus, it can be seen that the capacitance is higher at lower frequencies, due to the higher contribution to the capacitance from the ions in the blood plasma. Further, it can be seen that blood plasma has a higher capacitance than blood with 65% haematocrit at a given frequency. This is because blood cells effectively reduce the ionic concentration while having a relatively low capacitance themselves.

FIG. 4b shows the corresponding Bode plot for the real capacitance C′, and FIG. 4c) shows the corresponding Bode plot for the imaginary capacitance C′, where the relaxation R_{f} frequency is indicated by the minimum plateau for C″.

The frequency at which charging/discharging is harmonic with the oscillation of the potential is known as the relaxation frequency R_{f} and indicates the interfacial capacitance of the system Cᵢ. It is not necessary to determine the relaxation frequency R_{f} in the present method.

The “semicircle” in the Nyquist plot of FIG. 4a ends exactly at the relaxation frequency, R_{f} (see also FIG. 4c), and it gives the projection where the graph should touch the real capacitance C′ axis, which is the interfacial capacitance Cᵢ of the system. In this plasma/blood cell system, Cᵢ is dominated by charging/discharging of ions in the plasma and R_{f} is around 10² Hz, which is considered to be a low frequency. Without being constrained by theory, at high frequencies (e.g. above 10⁵ Hz) the oscillation of the input signal is sufficiently fast that the rearrangement of ions does not happen. The capacitance therefore becomes dominated by oscillation of the dipole of polarizable species. Thus, when measuring the capacitance of blood at high frequencies (frequencies tending to infinity) the contribution to the output signal from ions and other ionic species becomes insignificant. Accordingly, the inventors have identified that by calculating the capacitance of a blood sample at high frequencies that are relatively insensitive to the ionic strength of the blood, haematocrit can be calculated with much lower error introduced by varying blood salt concentrations. Note that at high frequencies the capacitance signal increases with the increase of haematocrit (see FIG. 5b), as predicted by the following equation:

$${k = [ ( 2 {e}^{2} N ) / {ε}_{r} {ε}_{0} {k}_{B} T ) ]}^{1 / 2}$$

where N is the molar concentration of red blood cells.

FIGS. 5a-5d illustrate haematocrit detection performance for fixed sodium concentration using the above described ‘capacitance’ method and the conventional Maxwell-Fricke method. Figure Sa is a Nyquist plot of capacitance fitted circular arcs for a range of blood samples with a haematocrit of 0 and 20 to 65%, in 5% increments, without any additional ions added to the blood sample (i.e. a Na⁺ spike, discussed further below, is 0 mM). As with FIG. 4a, it can be seen that at low frequencies the capacitance decreases with increasing haematocrit, as expected.

FIG. 5b is a calibration plot (derived from the data of FIG. 5a) demonstrating observed haematocrit ϕ versus estimated intercept of the axis of the real component of the complex capacitance C′_{intercept}, including the linear regression slope (black line) and 95% prediction interval (grey shading).

FIG. 5c is the known haematocrit ϕ versus haematocrit recovered using the Maxwell-Fricke method for a range of blood samples, including the linear regression slope (solid line) and the 1:1 line (dashed line). FIG. 5d is as in FIG. 4c, except that haematocrit ϕ is recovered via the C′_{intercept} calibration of FIG. 5b i.e. using the capacitance method of the present disclosure. As can be seen from comparison of FIGS. 5c and 5d, the haematocrit accuracy at fixed sodium concentration of the capacitance method is superior to the Maxwell-Fricke method. FIG. 5f is a plot of observed haemoglobin concentration versus haemoglobin concentration recovered via the C′_{intercept} calibration (capacitance method), including the linear regression slope (solid line). Thus, it can be seen that the capacitance method can also be used to determine haemoglobin concentration in blood with reasonable accuracy.

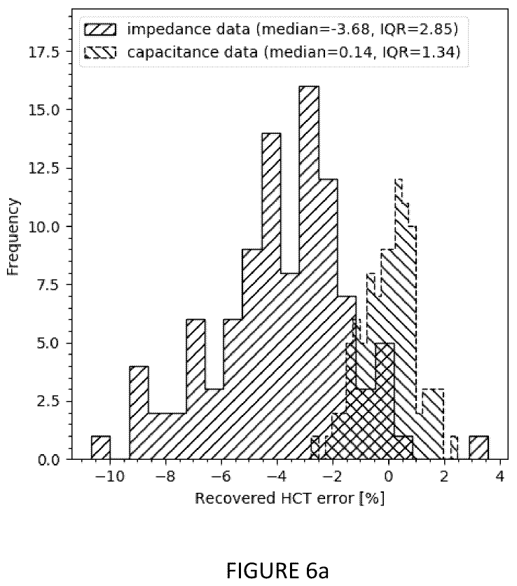

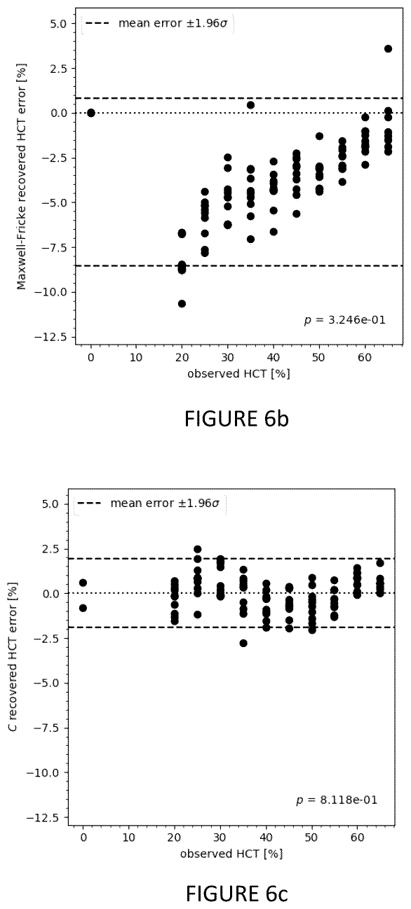

FIG. 6a is a series of histograms demonstrating the distribution of haematocrit ϕ recovery error for the Maxwell-Fricke (impedance) and C′_{intercept} calibration (capacitance) methods. As can be seen, the error is much lower when using the capacitance method. FIG. 6b is haematocrit ϕ recovery error from the Maxwell-Fricke method versus observed haematocrit ϕ, with the mean error +/−1.96 standard deviations denoted (dashed lines) alongside the p-value from a D'Agostino and Pearson normality test. FIG. 6c is as in FIG. 6b, except that haematocrit ϕ recovery errors are from the C′_{intercept} calibration method. Again it can be seen that the error is much lower when using the capacitance method.

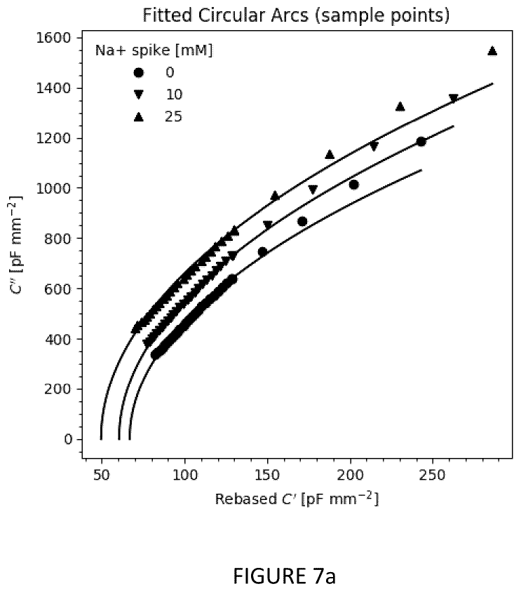

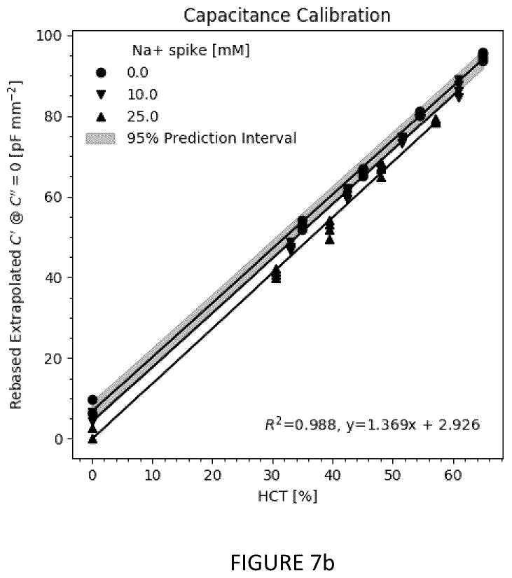

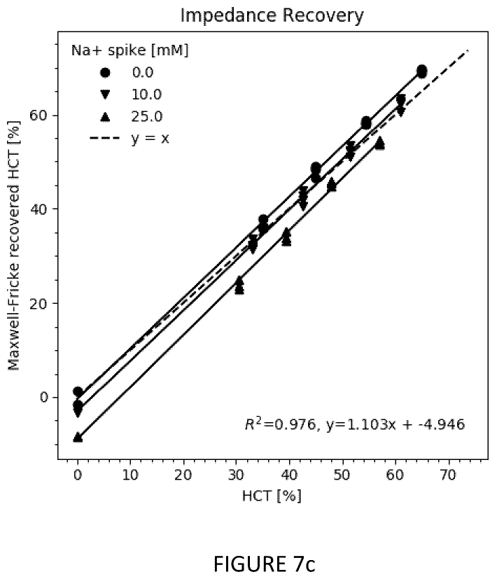

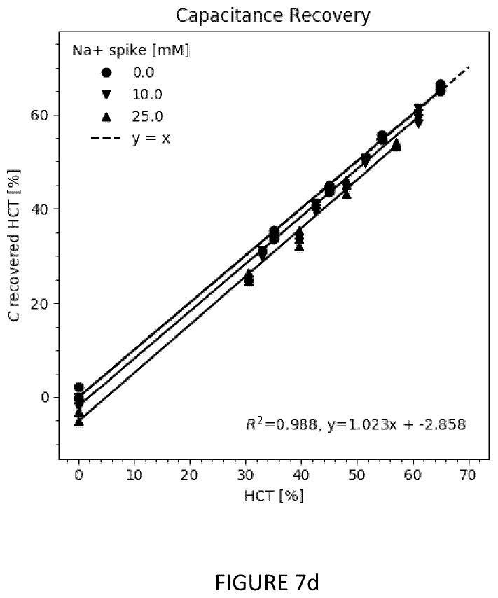

FIGS. 7a-d are as in FIG. 5, expect for varying Na⁺ concentration spikes. FIG. 7a shows capacitance data for blood with fixed haematocrit and Na⁺ spikes of 0.0, 10.0 and 25.0 mM.

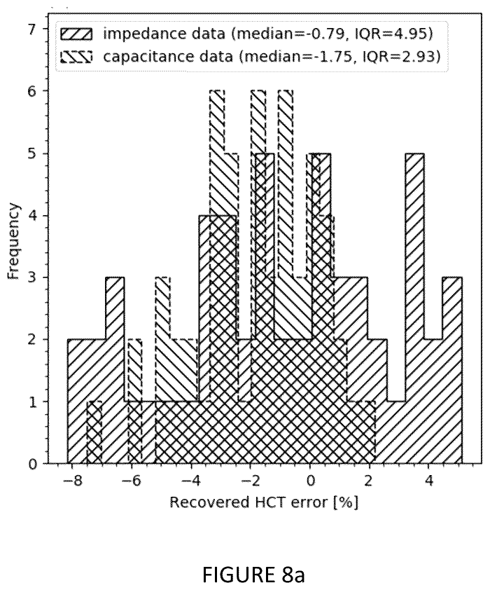

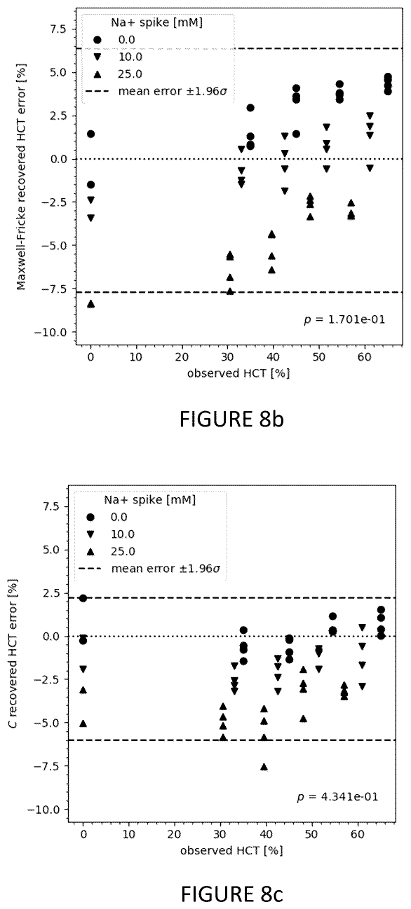

FIGS. 8a-c are as in FIGS. 6a-c, expect for varying Na⁺ concentration spikes. As can be seen, recovered haematocrit error is much lower when using the capacitance method instead of the impedance method. Thus, it follows that the capacitance method is less sensitive to variations in blood plasma conductivity/ionic strength than impedance methods.

### Haematocrit Sensor

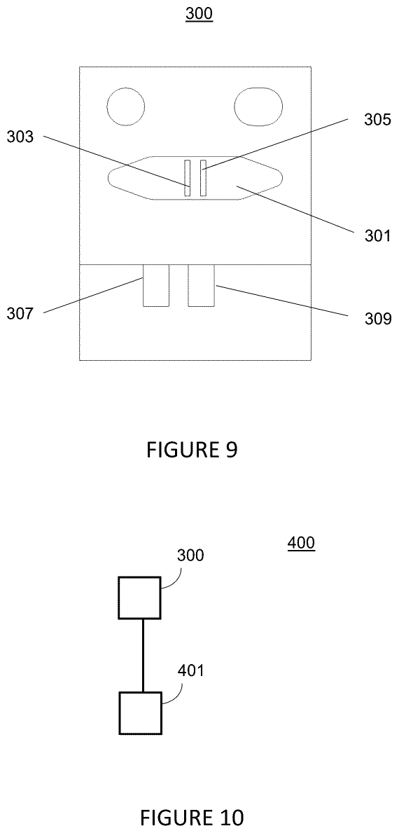

FIG. 9 illustrates a cell 300 for performing an impedance measurement on a blood sample.

The cell 300 comprises a chamber 301 for receiving a blood sample. The chamber 301 comprises two electrodes 303, 305. The electrodes may be made of platinum, gold, glassy carbon or any other suitable electrode material. The chamber 301 may comprise more than two electrodes 303, 305, for example the chamber may comprise a reference electrode. A distance between the electrodes 303, 305 is 0.6 mm. When the two electrodes are submerged in blood plasma, a solution resistance of the blood plasma is less than 5 kΩ. The cell further comprises two electrode contacts 307, 309. Each electrode 303, 305 is connected to a respective one of the electrode contacts 307, 309 via a track (not illustrated). The resistance of each track is 0.7 kΩ.

FIG. 10, illustrates a device 400 configured to determine haematocrit of a blood sample. The device comprises the cell 300 of FIG. 9 in electrical communication with a potentiostat 401 configured to determine a plurality of complex impedance values of a blood sample in the cell 300. The cell 300 may also be incorporated into the system of FIG. 3, i.e. by replacing the cell 201.

The traditional haematocrit sensor design for resistance-based systems such as the Maxwell-Frick system uses two parallel conductive electrodes optimised to have a high solution resistance to allow for greater difference in signal between low and high blood resistance. The inventors have identified that this is approach is not optimal for the capacitance methods disclosed above and produce a calibration curve with low gradient response from blood samples. By removing as much resistance from the electrode design in terms of track resistance (for example by using tracks with greater cross-sectional area) and solution resistance (for example by using electrodes with the closest possible spacing), performance can be optimised.

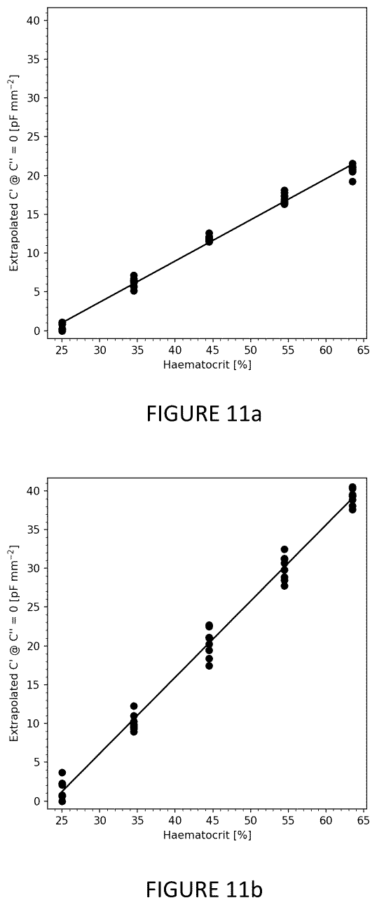

FIGS. 11a and 11b show the effect of optimising the cell for the capacitance method, most notably showing an improvement of approximately twice the gradient in the calibration curve which then improves the separation between datapoints.

FIG. 11a shows a calibration curve for the capacitance method using an electrode spacing of 2.4 mm. The equation of line is y=0.311x−12.9 with an R\hat{ }2 value of 0.9940. FIG. 11b shows a calibration curve for the capacitance method using an optimised cell design with electrode spacing of 0.6 mm. The equation of line is y=0.609x−36.1 with an R\hat{ }2 value of 0.9935.

The embodiments of the invention shown in the drawings and described above are exemplary embodiments only and are not intended to limit the scope of the appended claims, including any equivalents as included within the scope of the claims. Various modifications are possible and will be readily apparent to the skilled person in the art. It is intended that any combination of non-mutually exclusive features described herein are within the scope of the present invention. That is, features of the described embodiments can be combined with any appropriate aspect described above and optional features of any one aspect can be combined with any other appropriate aspect.

1. A method of determining haematocrit or haemoglobin concentration of a blood sample, the method comprising:
calculating, based on a plurality of complex capacitance values of the blood sample, a complex capacitance at a predetermined imaginary component value, each of the plurality of complex capacitance values of the blood sample having a corresponding frequency; and
determining the haematocrit or haemoglobin concentration based on the calculated complex capacitance or a real component of the calculated complex capacitance.

2. The method according to claim 1, wherein calculating a complex capacitance at a predetermined imaginary component value comprises extrapolating from the plurality of complex capacitance values.

3. The method according to claim 2, wherein extrapolating comprises fitting a circular arc to the plurality of complex capacitance values in capacitance space and extrapolating the arc to calculate the complex capacitance at the predetermined imaginary component value, optionally wherein the circular arc fitting is transformed to a weighted ordinary least squares fitting.

4. The method according to claim 3, wherein the corresponding frequencies, f, have a maximum value, fₘₐₓ, and the arc is fit for the range of frequencies f≤fₘₐₓ.

5. The method according to claim 3, wherein the corresponding frequencies, f, have a minimum value, fₘᵢₙ, and the circular arc is fit for the range of frequencies fₘᵢₙ≤f.

6. The method according to claim 3, wherein the circular arc is fit by applying an algorithm for least-squares estimation of nonlinear parameters.

7. The method according to claim 1, wherein determining the haematocrit or haemoglobin concentration based on the calculated complex capacitance or the real component of the calculated complex capacitance comprises using a calibration curve.

8. The method according to claim 1, wherein the calculated complex capacitance or the real component of the calculated complex capacitance corresponds to the capacitance of the blood sample at frequencies tending towards infinity.

9. The method according to claim 1, wherein each corresponding frequency of the plurality of complex capacitance values of the blood sample is less than or equal to 1000 kHz.

10. The method according to claim 1, wherein each corresponding frequency of the plurality of complex capacitance values of the blood sample is greater than or equal to 0.1 Hz.

11. The method according to claim 1, wherein the predetermined imaginary component value is less than or equal to 500 pF/mm2, preferably less than or equal to 200 pF/mm2, more preferably less than or equal to 100 pF/mm2, and yet more preferably substantially equal to or equal to zero.

12. The method of determining haematocrit or haemoglobin concentration of a blood sample, the method comprising:
determining a plurality of complex impedance values of the blood sample, each of the plurality of complex impedance values having a corresponding frequency;
calculating a plurality of complex capacitance values of the blood sample based on the plurality of complex impedance values; and
performing a method according to any preceding claim, using the plurality of complex capacitance values.

13. The computer-readable medium comprising instructions which, when executed by one or more processors, causes the one or more processors to perform the method of claim 1.

14. A system for determining haematocrit or haemoglobin concentration of a blood sample, the system comprising:
a cell configured to receive the blood sample;
a device configured to determine a plurality of complex impedance values of the blood sample;
the computer-readable medium according to claim 13; and
a processor configured to execute the instructions of the computer-readable medium.

15. The device configured to determine haematocrit of a blood sample, the device comprising two electrodes, wherein a distance between the electrodes is less than or equal to 2 mm.

16. The device according to claim 15, wherein the two electrodes are arranged such that when the electrodes are submerged in blood plasma, a solution resistance of the blood plasma is less than 20 kΩ.

17. The device according to claim 15, wherein a track resistance of a track between one of the electrodes and a corresponding electrode contact is less than or equal to 1 kΩ.
